(19) United States
(12) Patent Application Publication
(10) Pub. No.: US 2012/0014888 A1
(43) Pub. Date: Jan. 19, 2012

(54) Matte Skin Finish Compositions

(75) Inventors: Limin Liu, Palatine, IL (US); Ralph Spindler, Palatine, IL (US); Kevin Cureton, Evanston, IL (US)

(73) Assignee: AMCOL INTERNATIONAL CORPORATION, Hoffman Estates, IL (US)

(21) Appl. No.: 13/145,490

(22) PCT Filed: Jan. 25, 2010
(86) PCT No.: PCT/US10/21983

§ 371 Date: Oct. 3, 2011

Related U.S. Application Data
(60) Provisional application No. 61/148,171, filed on Jan. 29, 2009.

(51) Int. Cl. A61K 8/30; A61K 8/06; A61Q 17/04; A61Q 19/02; A61Q 15/00

(52) U.S. Cl. 424/59; 424/62; 424/65; 424/69

(57) ABSTRACT

A composition and method for reducing the shine of human skin is disclosed. The composition imparts a topical matte finish to the skin for an extended time period. The composition contains polymeric microparticles, optionally loaded with a cosmetic or therapeutic skin care compound.

### CROSS REFERENCE TO RELATED APPLICATIONS

This application claims the benefit of U.S. provisional patent application No. 61/148,171, filed Jan. 29, 2009, incorporated herein by reference in its entirety.

### FIELD OF INVENTION

The present invention relates to skin care compositions. More specifically, the present invention relates to skin care and cosmetic compositions that reduce the appearance of shiny or oily skin and impart a topical matte finish when applied to human skin.

### BACKGROUND OF THE INVENTION

An undesirable skin condition is “oily skin,” which results from an excessive amount of sebum on the skin. Human skin naturally manufactures and secretes sebum from sebaceous glands located near the skin surface. Sebum includes fatty acids, esters, glycerides, and other endogenous lipids, which lubricate and protect skin against moisture loss by forming a film over the surface of the skin. Along with natural moisturizers in the epidermis, sebum helps to keep the skin soft and hydrated.

A build-up of sebum on the surface of skin can cause the skin to appear shiny or oily. In addition to a visually unappealing appearance of shiny or oily skin, sebum is associated with a disagreeable tactile sensation, and sebum build-up also can highlight skin imperfections. Freshly secreted sebum has some antibacterial properties and is not harmful. However, in cases of excess secretion, the sebum can combine with cell debris and pollutants to form waxy plugs or comedones that block pores and encourage bacterial colonization. Comedones are implicated in some forms of acne. Excess secretion of the sebum also may be a factor causing makeup to come off which leads to, for example, a “shiny” or “drab” appearance of the skin, or an “unevenness”, “rumpling”, or “disappearance” of the makeup itself.

Oily skin affects various age groups. People having oily skin often suffer social embarrassment for lacking a dry looking skin. Oily skin can be an especially difficult problem during the day when the oil builds up, and in situations where it is difficult to repeatedly wash the skin to prevent shine build up. Additionally, for many people with oily skin, skin problems such as acne are exacerbated by the excessive oil content, causing a more difficult treatment of the condition.

Current methods used to reduce the amount of sebum on the surface of skin include increasing the consumption of water, applying moisturizers to skin, and using compositions to absorb sebum from skin. Skin care compositions that absorb the excess sebum on the skin, diffuse or scatter light, provide a matte finish, and help hide fine lines and blemishes are desirable. Compositions including a high level of pigments typically have been used to achieve such a matte effect. But a high pigment level provides a heavy, cakey product that leaves a heavy, dull color on skin. In addition, such a matte effect usually does not last for extended periods.

In particular, cleansing lotions, facial washes, and various skin care products can be widely used to address the esthetic problems associated with oily skin. Skin care products that are used for covering up oily skin can be traditional adsorbents, such as talcum powder and clays, like bentonite. Such a treatment also dries and deoils the skin by absorbing and removing natural lubricants and moisture. Usually, the powders and/or anti-sebum components are incorporated into liquids, aqueous creams, and gel like compositions that may contain alcohol to give the skin a matte appearance. However, the compositions as a whole may simply dehydrate the skin and mop up oily secretions for a brief time after application.

The development of cosmetic products that can be free of color components, and that provide an extended matte skin finish, has been especially challenging. Researchers have explored different materials to provide a long lasting matte finish using skin care products, such as foundations, lip sticks, daily use moisturizers, and many other similar products. However, cosmetic and skin care compositions that provide a matte finish for six hours or longer are rare, especially leave-on compositions that do not contain color components and do not dehydrate the skin.

### SUMMARY OF THE INVENTION

The present invention is directed to cosmetic and skin care compositions used in methods of imparting a matte finish to the skin. More particularly, the present invention is directed to compositions that demonstrate an enhanced ability to impart a matte finish to skin through the use of polymeric microparticles. The compositions also absorb sebum from the skin.

Therefore, one aspect of the present invention is to provide a composition comprising about 1% to about 10%, by weight, polymeric microparticles. The polymeric microparticles can be used as is, or loaded with a skin care, therapeutic, or cosmetic agent to impart a benefit to the skin in addition to a matte finish.

Another aspect or the present invention is to provide a method or imparting a matte finish to skin comprising topically applying a composition comprising about 1% to about 10%, by weight, polymeric microparticles to a skin surface. The method is capable of imparting a matte finish for six hours or longer.

Yet another aspect of the present invention is to provide a composition and method that absorb sebum from the skin, while reducing the appearance of shiny or oily skin.

These and other aspects and novel features of the present invention will become apparent from the following detailed description of the preferred embodiments.

### BRIEF DESCRIPTION OF THE FIGURES

FIGS. 1 and 2 are plots of Delta Matte Index/Matte Index vs. Time (Hrs) comparing the composition of Example 2 to the commercial OC Eight product for Expert Graders and Panelists, respectively.

### DETAILED DESCRIPTION OF THE PREFERRED EMBODIMENTS

Consumers are continually seeking a product that can improve the visual appearance of their skin. Often, skin overproduces sebum which can cause skin to appear oily or shiny and have a slippery feel. The build-up of sebum also can result from normally functioning sebaceous glands in instances where the skin has not been washed for an extended period of time. Besides the visually unappealing appearance of shiny or oily skin, sebum build-up also can highlight skin imperfections and promote the development of skin acne.

A variety of oil adsorbing/absorbing materials are known in the art of cosmetic and skin care compositions. A problem demonstrated by many of these compositions is that it is not sufficient to simply soak up excess oil on the surface of the skin. The composition also should make the skin appear matte. Further, if composition components are not properly dispersed within the composition, or are used at too high of a concentration, the composition leaves a heavy, unpleasant feel during application, the composition may not appear uniform on the skin, and the composition or its components may flake off the skin yielding a very unsightly appearance. A matte finish on the skin is very important because it also gives the consumer an esthetic signal that the composition is still performing its intended function.

In accordance with the present invention, it has been found that polymeric microparticles overcome the above-identified problems and provide a solution to the shiny skin problem, including imparting a persistent matte finish to the skin. One class of polymeric microparticles, sold under tradename of POLY-PORE® by AMCOL International Corporation, Hoffman Estates, Ill. (INCI name of allyl methacrylates crosspolymer) is disclosed in U.S. Pat. Nos. 5,677,407; 5,712,358; 5,777,054; 5,830,967; 5,834,577, 5,955,552; and 6,107,429, each incorporated herein by reference. Additional polymeric microparticles for use in the present invention are sold by AMCOL International Corporation under the tradenames POLYTRAP® (INCI name of lauryl methacrylate/glycol methacrylate crosspolymer), disclosed in U.S. Pat. Nos. 4,962,170; 4,948,818; and 4,962,133, each incorporated herein by reference and MICROSPONGE® (methyl methacrylate/glycol dimethacrylate crosspolymer), disclosed in U.S. Pat. Nos. 4,690,825; 5,073,365; 5,135,740; 5,145,675; and 5,891,470, each incorporated herein by reference. Poly-HIPE polymers (e.g., a copolymer of 2-ethylhexyl acrylate, styrene, and divinylbenzene) available from Biopore Corporation, Mountain View, Calif., also are useful in the present invention. These polymeric microparticles are highly crosslinked copolymers, and are insoluble in water, hydrophilic organic solvents, and hydrophobic liquids. The polymeric microparticles typically exhibit a high absorbability for both hydrophobic and hydrophilic skin care agents. These polymeric microparticles also are able to adsorb both skin oil and perspiration.

In particular, adsorbent polymer microparticles prepared by a suspension polymerization technique, e.g., POLY-PORE® E200, are a highly porous and highly crosslinked polymer (i.e., contain greater than 80 mol %, preferably more than 90 mol %, and up to 100% mol. polyunsaturated monomers, such as allyl methacrylate, ethylene glycol dimethacrylate, and similar compounds containing at least two carbon-carbon double bonds) in the form of open (i.e., broken) spheres and sphere sections characterized by a mean unit particle size of about 0.5 to about 3,000 microns, preferably about 0.5 to about 300 microns, more preferably about 0.5 to about 100 microns, and most preferably about 0.5 to about 80 microns. A significant portion of the spheres is about 20 microns in diameter.

The polymeric microparticles are oil and water adsorbent, and have an extremely low bulk density of about 0.008 gm/cc to about 0.1 gm/cc, preferably about 0.009 gm/cc to about 0.07 gm/cc, and more preferably about 0.0095 gm/cc to about 0.04-0.05 gm/cc. The microparticles are capable of holding and releasing oleophilic (i.e., oil soluble or dispersible), as well as hydrophilic (i.e., water soluble or dispersible), agents, individually, or both oleophilic and hydrophilic compounds simultaneously.

The adsorbent polymer microparticles prepared by the suspension polymerization technique include at least two polyunsaturated monomers, preferably allyl methacrylate and an ethylene glycol dimethacrylate, and, optionally, monounsaturated monomers. The microparticics are characterized by being open to their interior, due either to particle fracture upon removal of a porogen after polymerization or to subsequent milling. The microparticles have a mean unit diameter of less than about 50 microns, preferably less than about 25 microns, and have a total adsorption capacity for organic liquids, e.g., mineral oil, that is at least about 72% by weight, preferably at least about 93% by weight, and an adsorption capacity for hydrophilic compounds and aqueous solutions of about 70% to about 89% by weight, preferably about 75% to about 89% by weight, calculated as weight of material adsorbed divided by total weight of material adsorbed plus dry weight of polymer. In a preferred embodiment, the broken sphere microparticles are characterized by a mean unit diameter of about 1 to about 50 microns, more preferably of about 1 to about 25 microns, most preferably, of about 1 to about 20 microns.

Preferred polymeric microparticle delivery systems comprise a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, and mixtures thereof. Specific examples of polymeric microparticles useful in the present invention can be the previously described POLY-PORE® E200, POLY-PORE® L200, POLYTRAP® 6603, POLYTRAP® 7603, MICROSPONGE® entrapments, and Poly-HIPE particles, used individually or in any combination.

Surprisingly, it has been found that polymeric microparticles impart a long-lasting matte finish on the skin when formulated into a composition at an appropriate level. In addition, the composition also provides a smooth, powdery, dry skin feel during and after the application of the product. If the amount of microparticles in the composition is too h h, e.g., greater than about 10%, by weight of the composition, or the composition is not formulated properly, the composition produces a non-uniform film on the skin (i.e., is blotchy) or a film that flakes off the skin. Also, if the amount of microparticles in the composition is too low, e.g., less than about 1%, by weight of the composition, the composition will adsorb skin oil for an extended period of time, but there is an insufficient amount of microparticles to impart a matte finish on the skin, and therefore the consumer will have a perception that the composition is not working. Preferred amounts of the polymeric microparticles in the matte skin finish composition are about 1% to about 8%, by weight. However, polymeric microparticle weight amounts of 1%, 2%, 3%, 4%, 5%, 6%, 7%, 8%, 9%, and 10%, and all ranges and subranges between about 1% and about 10%, by weight, are useful in the present invention.

A skin care or cosmetic composition containing the polymeric microparticles, loaded with an active agent or unloaded, can be a dispersion, an aqueous gel, anhydrous gel, water-in-oil emulsion, and/or oil-in-water emulsion. Clinical studies show that the matte finish lasts on the skin for six hours or longer.

The polymeric microparticles can be used “as is” in a present matte skin finish composition. Optionally, the polymeric microparticles can be loaded with one or more cosmetic and/or therapeutic compound prior to being formulated into a matte finish composition. The loaded or entrapped microparticles still effectively impart a matte finish due to the high capacity of the microparticles to adsorb large amounts of different materials simultaneously. For example, when a polymeric microparticle, like POLY-PORE® E200, first is loaded with 50%, by weight, salicylic acid, the microparticles still can absorb up to 10 times their weight in excess skin oil before being saturated. This is a significant advantage because it is possible to both deliver a skin care agent or drug to the skin and impart a matte skin finish simultaneously.

The other polymeric microparticles disclosed above have a similar ability although the amount of skin oil that can be adsorbed varies. For example, MICROSPONGE® particles are able to adsorb up to two times their weight of oil, even when the microparticles are previously loaded with a cosmetic or therapeutic compound, because the microparticles have an excess capacity to provide a matte finish for an extended time period of several hours. Additionally, it is possible to utilize a mixture of the different polymeric microparticle classes disclosed above, in either an unloaded or a loaded form, to optimize matte performance and the delivery of cosmetic and therapeutic skin care compounds. The microparticles can be loaded with a wide variety of skin care agents, esthetic agents, or topically active drugs.

Loading the polymeric microparticles with a cosmetic or therapeutic agent can be accomplished by spraying or adding the agent either directly to the microparticles in such a manner that an essentially homogeneous distribution of the agent is achieved on all the microparticles. Alternatively, if the agent is a solid compound, the agent first is dissolved in a suitable volatile solvent, the resulting solution is added to the microparticles, and then the volatile solvent is removed under vacuum with optional gentle heating. In some cases, this process is repeated several times to achieve a desired loading level of the agent. Another method of loading a solid agent that is not readily soluble in a volatile solvent is to first disperse the solid in a suitable carrier, such as polyether or polyol, and then add the dispersion directly to the polymeric microparticles.

The load of the agent in and on the polymeric microparticles can be 0%, or about 0.01%, to about 80 wt. %, when the agent is a solid material at room temperature (i.e., about 23° C. to 25° C.) or in a preferred amount of about 0.01% to about 50 wt. %. In every case, the polymeric microparticles optionally are loaded in an amount less than saturation such that the microparticles retain a capacity to absorb skin oils. The composition can contain microparticles that are loaded with a cosmetic or therapeutic agent, microparticles that are not loaded, or a mixture thereof.

Cosmetic and therapeutic compounds that can be loaded onto the polymeric microparticles include, but are not limited to, cyclomethicone, dimethicone, mineral oil, petrolatum, salicylic acid, benzoyl peroxide, tretinoin, sulfur, resorcinol, retinol and other retinoids, hydroquinone and other skin lightening agents, like kojic dipalmitate, dark circle reduction agents, slimming agents, antifungal agents, and antibacterial agents. In some preferred embodiments, anti-acne agents, like salicylic acid, benzoyl peroxide, adapalene, dapsone, clindamyacin, benzamyacin, azelaic acid, and tretinoin, are loaded onto the polymeric microparticles.

Retinoids that can be loaded onto, or entrapped by, the polymeric microparticles include, both naturally occurring and synthetic compounds having the general structure of vitamin A (retinol) and variations of that structure having similar biological and pharmacological activity as retinol. Examples of retinoids include, but are not limited to, retinol, retinal, retinyl acetate, retinyl palmitate, retinoic acid, retinyl propionate, retinyl linoleate, dehydroretinol, eretinate, eretrin, motretinide, and mixtures thereof. U.S. Pat. No. 5,851,538, incorporated herein by reference, discloses several additional useful retinoids.

The following are examples of matte skin finish compositions of the present invention.

### EXAMPLE 1

### Matte Finish Water-in-Oil Emulsion

| Ingredients (INCI Names) | Wt % |
|---|---|
| Water | 48.30 |
| Cyclopentasilicone (and) Cyclohexasilicone | 16.38 |
| Isopropyl Myristate | 11.96 |
| Methyl Methacrylate/Glycol Dimethacrylate | 8.00 |
| Crosspolymer | |
| Butylene Glycol | 4.60 |
| PEG-150 | 4.60 |
| Cetyl PEG/PPG-10/1 Dimethicone | 2.76 |
| Sodium Chloride | 0.92 |
| Benzyl Alcohol | 0.92 |
| Phenoxyethanol | 0.83 |
| Bis-PEG-12 Dimethicone Beeswax | 0.46 |
| Hydrogenated Castor Oil | 0.18 |
| Disodium EDTA | 0.09 |

### EXAMPLE 2

### Matte Finish Oil-in-Water Emulsion

| Ingredients (INCI Names) | Wt % |
|---|---|
| Water | 69.72 |
| Methyl Methacrylate/Glycol Dimethacrylate | 8.00 |
| Crosspolymer | |
| Caprylic/Capric Triglyceride | 7.36 |
| Dimethicone | 4.41 |
| Cetearyl Alcohol | 2.82 |
| Glycerin | 2.76 |
| Glyceryl Stearate | 0.92 |
| PEG-100 Stearate | 0.92 |
| Benzyl Alcohol | 0.92 |
| Phenoxyethanol | 0.83 |
| Ceteareth-20 | 0.49 |
| Magnesium Aluminum Silicate | 0.46 |
| Myristyl Myristate | 0.37 |
| Xanthan Gum | 0.02 |

### Measurement of the Matte Finish/Shine Reduction Effect

The matte finish effect was evaluated by comparing the composition of Example 2 to a leading commercial product (OC Eight, Ferndale Laboratories) using an in vivo matte finish study.

Twenty subjects having oily skin were recruited after having refrained from using any oil control products for at least one week. The subjects were tested with an unwashed face. No cosmetics or any other face products were applied after washing the face the night before the study. The subjects also refrained from using any oil control products during the study. The same subjects were used for both parts of the study. For one part of the study, the first composition was applied to one side of the forehead, then after waiting at least three days, the second composition was applied to the opposite side of the forehead. About 2.0 mg/cm² of a composition was applied on a 4×5 cm² area of the forehead. This was a half side study. The identity of the compositions was blinded from the study director. The matte finish composition of Example 2 was applied to the left side of the forehead, with the control side being the right side of the forehead. The OC Eight product was applied to the right side of the forehead with the control side being the left side of the forehead. Both the panelists and trained technicians (expert graders) scored the matte appearance using the ordinal scale (1=very shiny; 5=moderate shiny, 10=very matte).

[00]31 Photographs also were taken to record the matte finish effect. A Visia Digital Imaging System (Canfield Imaging Systems, Fairfield, N.J.) was used to take digital photographs by parallel-polarized lighting. A standard chin rest and a three-point adjustable head support ensured proper positioning of the panelist for each time point.

Evaluations (photographs, and panelist and expert grading) were completed at baseline and at 30 minutes, 1 hour, 2 hours, 3 hours, 4 hours, 5 hours, and 6 hours post application.

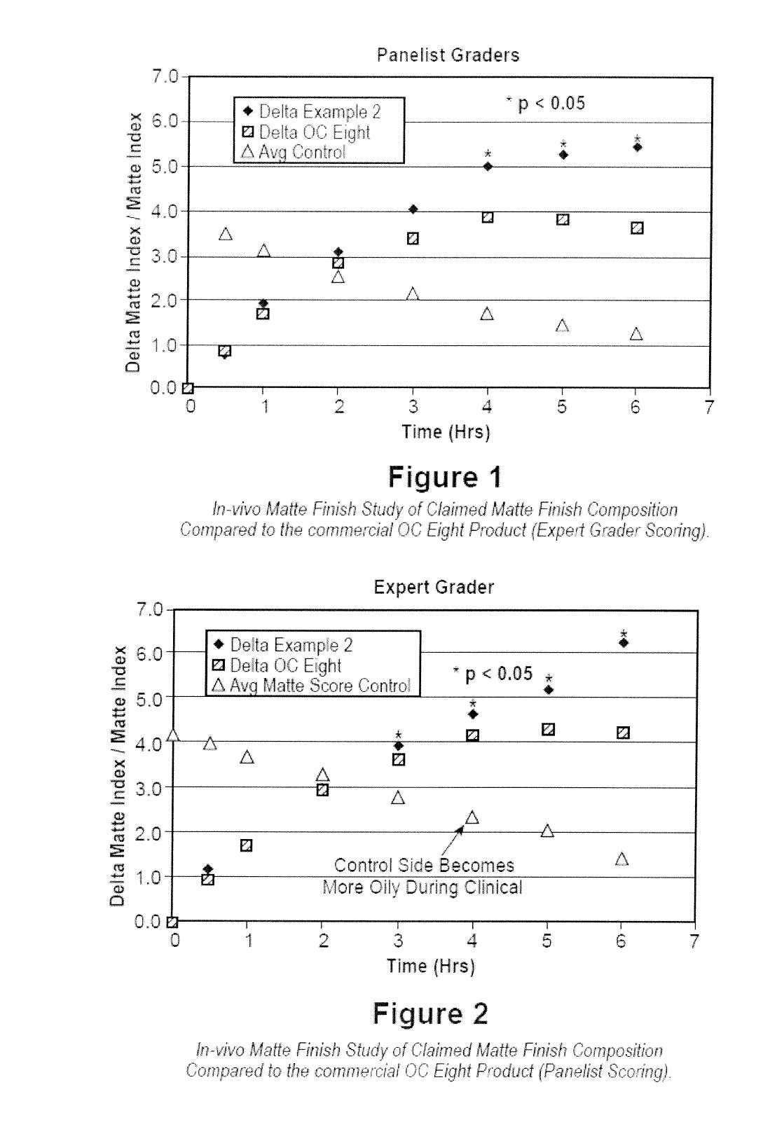

The results are summarized in Table 1 (Expert Score) and Table 2 (Panelist Score), and illustrated in FIGS. 1 and 2. For the expert grader scores, the composition of Example 2 showed increasing matte index scores throughout the clinical study, and, using a T Test to statistically compare the two results, showed that for hours 3-6, the scores for the composition of Example 2 were statistically greater than the scores for the commercial control product (p<0.05). The data clearly show that a present composition imparts a better matte finish than the leading present day commercial product (OC Eight).

Table 2 summarizes the results from the panelists self evaluation of the matte index. Like the expert scores, the panelists saw continued improvement for both products over the course of the study, except for the last timepoint for the commercial control where the score decreased. A statistical analysis of the data showed that at hours 4-6, the composition of Example 2 had a statistically improved score over the commercial control. The importance of the panelist score is that in order for a consumer to believe that a matte finish product is working, the consumer must be able to see that the composition is performing by maintaining a matte finish on the skin.

This data also is plotted graphically in FIGS. 1 and 2 wherein the change from baseline was calculated by subtracting either the Expert Grader or Panelist scores from the baseline score for each timepoint. The average control scores from both phases of the clinical trial also arc shown to emphasize that the skin of all panelists continued to show increased oiliness during the study. As presented in the Tables, the results clearly show that (a) the composition of Example 2 provides a matte finish at six hours, (b) the scores continue to improve in the case of the expert graders, and (c) the product has the capacity to continue to provide a matte finish for even a longer period of time while remaining cosmetically elegant.

TABLE 1
Numeric scores from Clinical Trial, Expert Scores

| Example 2 | | | | OC Eight | | | |
|---|---|---|---|---|---|---|---|
| | | | Example 2, | | | OC Eight, | |
| | Expert | | Change | Expert | | Change | Average |
| | Score, | Control | from | Score, | Control | from | Control |
| Time | Example 2 | Score | Control | OC Eight | Score | Control | Scores |
| 0 | 4.1 | 4.1 | 0.0 | 4.3 | 4.3 | 0.0 | 4.2 |
| 0.5 | 5.2 | 4.0 | 1.2 | 5.1 | 4.1 | 1.0 | 4.0 |
| 1 | 5.6 | 3.8 | 1.8 | 5.3 | 3.6 | 1.7 | 3.7 |
| 2 | 6.5 | 3.5 | 3.0 | 6.0 | 3.1 | 3.0 | 3.3 |
| 3 | 6.8 | 2.9 | 3.9 | 6.3 | 2.7 | 3.6 | 2.8 |
| 4 | 7.1 | 2.5 | 4.6 | 6.4 | 2.2 | 4.2 | 2.3 |
| 5 | 7.3 | 2.2 | 5.2 | 6.1 | 1.8 | 4.3 | 2.0 |
| 6 | 7.6 | 1.4 | 6.2 | 5.6 | 1.4 | 4.2 | 1.4 |

TABLE 2
Numeric Score from Clinical Trial, Panelist Scores

| Example 2 | | | | OC Eight | | | |
|---|---|---|---|---|---|---|---|
| | | | Example 2, | | | OC Eight, | |
| | Panelists | | Change | Expert | | Change | Average |
| | Score, | Control | from | Score, | Control | from | Control |
| Time | Example 2 | Score | Control | OC Eight | Score | Control | Scores |
| 0 | 3.5 | 3.5 | 0.0 | 3.6 | 3.6 | 0.0 | 3.6 |
| 0.5 | 4.2 | 3.5 | 0.8 | 4.4 | 3.5 | 0.9 | 3.5 |
| 1 | 4.8 | 2.9 | 1.9 | 4.9 | 3.2 | 1.7 | 3.0 |
| 2 | 5.5 | 2.5 | 3.1 | 5.3 | 2.5 | 2.8 | 2.5 |
| 3 | 6.0 | 2.0 | 4.0 | 5.6 | 2.3 | 3.4 | 2.1 |
| 4 | 6.6 | 1.6 | 5.0 | 5.5 | 1.7 | 3.8 | 1.7 |
| 5 | 6.6 | 1.4 | 5.2 | 5.2 | 1.4 | 3.8 | 1.4 |
| 6 | 6.5 | 1.1 | 5.4 | 4.8 | 1.3 | 3.6 | 1.2 |

In accordance with an important feature of the present invention, a matte skin finish composition can contain any of a wide variety of topically applied compounds, either water soluble or oil soluble, for providing additional cosmetic or therapeutic effects. These additional compounds arc not necessarily loaded onto polymeric microparticles, but can be present in a free form in the composition, in a sufficient amount in either embodiment to perform their intended function. As used herein, the term “free form” of a compound means that the compound is not entrapped or loaded onto polymeric microparticles, but rather is dissolved or dispersed in the liquid or gel phase of the matte skin finish composition.

For example, a present composition also can contain a skin-lightening agent. Useful skin-lightening agents include, but are not limited to, skin exfoliants; hydroquinone or a derivative thereof, such as benzylhydroquinone ether; ascorbic acid or a derivative thereof, such as magnesium ascorbyl phosphate; a caffeic acid or ester thereof; a benzofuran, such as 5- or 6-hydroxybenzofuran; a plant extract, such as licorice, mulberry, heather, and angelica ashitaba; a pearl extract; a steroidal anti-inflammatory agent of the hydrocortisone-type and the like; a nonsteroidal anti-inflammatory agent selected from the group consisting of acetylsalicylic acid, acetaminophen, naproxen, and fenamic acid derivatives, such as the sodium salt; an anti-inflammatory agent, such as alpha-bisabolol, beta-glycyrrhetinic acid, allantoin, aloe extract, rosmarinic acid, azulene or a derivative thereof, asiaticoside, sericoside, ruscogenin, escin, escolin, quercetin, rutin, betulinic acid or a derivative thereof, catechin or a derivative thereof; and mixtures thereof.

The additional compound also can be one of, or a mixture of, a cosmetic compound, a medicinally active compound, a compound used in cosmetics or personal care, or any other compound that is useful upon topical application to the skin. Such topically active agents include, but are not limited to, skin-care compounds, plant extracts, antioxidants, insect repellants, counterirritants, vitamins, steroids, antibacterial compounds, antifungal compounds, anti-inflammatory compounds, topical anesthetics, sunscreens, optical brighteners, and other cosmetic and medicinal topically effective compounds.

For example, a skin conditioner can be the topically applied compound. Skin conditioning agents include, but are not limited to, humectants, such a fructose, glucose, glycerin, propylene glycol, glycereth-26, mannitol, urea, pyrrolidone carboxylic acid, hydrolyzed lecithin, coco-betaine, cysteine hydrochloride, glucamine, PPG-15, sodium gluconate, potassium aspartate, oleyl betaine, thiamine hydrochloride, sodium laureth sulfate, sodium hyaluronate, hydrolyzed proteins, hydrolyzed keratin, amino acids, amine oxides, water-soluble derivatives of vitamins A, E, and D, amino-functional silicones, ethoxylated glycerin, alpha-hydroxy acids and salts thereof, fatty oil derivatives, such as PEG-24 hydrogenated lanolin, and mixtures thereof Numerous other skin conditioners are listed in the CTFA Cosmetic Ingredient Handbook, First Ed., J. Nikotakis, ed., The Cosmetic, Toiletry and Fragrance Association (1988), (hereafter CTFA Handbook), pages 79-84, incorporated herein by reference.

The skin conditioner also can be a water-insoluble ester having at least 10 carbon atoms, and preferably 10 to about 32 carbon atoms. Suitable esters include those comprising an aliphatic alcohol having about eight to about twenty carbon atoms and an aliphatic or aromatic carboxylic acid including from two to about twelve carbon atoms, or conversely, an aliphatic alcohol having two to about twelve carbon atoms with an aliphatic or aromatic carboxylic acid including about eight to about twenty carbon atoms. The ester is either straight-chained or branched. Suitable esters, therefore, include, for example, but are not limited to:
(a) aliphatic monohydric alcohol esters, including, but not limited to:
myristyl propionate,
isopropyl isostearate,
isopropyl myristate,
isopropyl palmitate,
cetyl acetate,
cetyl propionate,
cetyl stearate,
isodecyl neopentanoate,
cetyl octanoate,
isocetyl stearate;
(b) aliphatic di- and tri-esters of polycarboxylic acid, including, but not limited to:
diisopropyl adipate,
diisostearyl fumarate,
dioctyl adipate, and
triisostearyl citrate;
(c) aliphatic polyhydric alcohol esters, including, but not limited to: propylene glycol dipelargonate;
(d) aliphatic esters of aromatic acids, including, but not limited to:
C₁₂-C₁₅ alcohol esters of benzoic acid,
octyl salicylate,
sucrose benzoate, and
dioctyl phthalate.
   Numerous other esters are listed in the CTFA Handbook, at pages 24 through 26, incorporated herein by reference. The skin conditioner also can be a silicone polymer, like a poly(dimethylsiloxane) over a wide range of molecular weights, e.g., dimethicone fluids of viscosity 1 to 3000 centipoises and dimethicone polyols.

An example of moisturizing Matte Finish cream is illustrated in Example 3.

### EXAMPLE 3

### A Matte Finish Hydration Cream

| Ingredients (INCI Name) | Wt. % |
|---|---|
| Water | 61.26 |
| Methyl Methacrylate/Glycol Dimethacrylate | 8.00 |
| Crosspolymer | |
| Caprylic/Capric Triglyceride | 7.36 |
| Dimethicone | 4.41 |
| Myristyl Myristate | 3.70 |
| Glycerin | 3.00 |
| Cetearyl Alcohol | 2.82 |
| Sodium Lactate | 2.50 |
| Sodium Gluconate | 2.50 |
| Glyceryl Stearate | 0.92 |
| PEG-100 Stearate | 0.92 |
| Benzyl Alcohol | 0.92 |
| Phenoxyethanol | 0.83 |
| Ceteareth-20 | 0.49 |
| Disodium EDTA | 0.10 |
| Sodium Hyaluronate | 0.10 |
| Allyl Methacrylates Crosspolymer | 0.10 |
| Magnesium Aluminum Silicate | 0.05 |
| Xanthan Gum | 0.02 |

The topically applied compound also can be an antioxidant or an optical brightener, like a distyrylbiphenyl derivative, stilbene or a stilbene derivative, a pyralozine derivative, or a coumarin derivative. Optical brighteners useful as the topically applied compound can be any compound capable of absorbing an invisible UV portion of the daylight spectrum, and converting this energy into the longer visible wavelength portion of the spectrum. The optical brightener is colorless on the substrate, and does not absorb energy in the visible part of the spectrum. The optical brightener typically is a derivative of stilbene or 4,4′-diaminostilbene, biphenyl, a 5-membered heterocycle, e.g., triazole, oxazole, or imidazole, or a 6-membered heterocycle, e.g., a coumarin, a naplithalamide, or an s-triazine.

The optical brighteners are available under a variety of tradenames, such as TINOPAL®, LEUCOPHOR®, and CALCOFLUOR®. Specific fluorescent compounds include, but are not limited to, TINOPAL® 5BM, CALCOFLUOR® CG, and LEUCOPHOR® BSB.

In addition, other compounds can be included in a present composition as the topically active compound in an amount sufficient to perform their intended function. For example, sunscreen compounds such as benzophenone-3, tannic acid, uric acids, quinine salts, dihydroxy naphtholic acid, an anthranilate, p-aminobenzoic acid, phenylbenzimidazole sulfonic acid, PEG-25, or p-aminobenzoic acid can be used as the topically applied compound. Further, sunscreen compounds such as dioxybenzone, ethyl 4-[bis(hydroxypropyl)]aminobenzoate, glyceryl aminobenzoate, homosalate, methyl anthranilate, octocrylene, octyl methoxycinnamate, octyl salicylate, oxybenzone, padimate O, red petrolatum, titanium dioxide, 4-menthylbenzylidene camphor, benzophenone-1, benzophenone-2, benzophenone-6, benzophenone-12, isopropyl dibenzoyl methane, butyl methoxydibenzoylmethane, zotocrylene, or zinc oxide can be used as the topically applied compound. Other sunscreen compounds are listed in CTFA Handbook, pages 86 and 87, incorporated herein by reference.

A matte finish sunscreen lotion of the present invention is provided in Example 4.

### EXAMPLE 4

### A Matte Finish Sunscreen

| Ingredients (INCI Names) | Wt % |
|---|---|
| Water | 50.88 |
| Homosalate | 10.56 |
| MICROSPONGE C111A (AMCOL)¹ | 5.52 |
| Ethylhexyl Salicylate | 4.80 |
| Methyl Methacrylate/Glycol Dimethacrylate | 4.00 |
| Crosspolymer | |
| Glycolic Acid | 3.86 |
| Glycerin | 3.84 |
| Butyl Methoxydibenzoylmethane | 2.88 |
| Octocrylene | 1.92 |
| Cetyl Alcohol | 1.92 |
| Ammonium Hydroxide | 1.51 |
| Glyceryl Stearate | 1.44 |
| Dimethicone | 0.96 |
| Methyl Glucose Sesquistearate | 0.96 |
| Potassium Cetyl Phosphate | 0.96 |
| Hydrogenated Palm Glycerides | 0.96 |
| Butylene Glycol | 0.88 |
| Phenoxyethanol | 0.86 |
| Panthenol | 0.29 |
| Tocopheryl Acetate | 0.24 |
| Bentonite | 0.19 |
| Xanthan Gum | 0.14 |
| Camellia Oleifera Leaf Extract | 0.13 |
| Disodium EDTA | 0.10 |
| Allantoin | 0.10 |
| Ethylhexylglycerin | 0.09 |

¹MICROSPONGE C111A is a methyl methacrylate/glycol dimethacrylate crosspolymer (and) glycolic acid (about 70%), commercially available from AMCOL Health and Beauty Solutions, Hoffman Estates, IL.

Similarly, topically applied drugs, like antifungal compounds, antibacterial compounds, anti-inflammatory compounds, topical anesthetics, skin rash, skin disease, and dermatitis medications, and antiitch and irritation-reducing compounds can be used as the active agent in the compositions of the present invention. For example, analgesics such as benzocaine, dyclonine hydrochloride, aloe vera, and the like; anesthetics such as butamben picrate, lidocaine hydrochloride, xylocaine, and the like; antibacterials and antiseptics, such as povidone-iodine, polyrnyxin b sulfate-bacitracin, zinc-neomycin sulfate-hydrocortisone, chloramphenicol, ethylbenzethonium chloride, erythromycin, and the like; antiparasitics, such as lindane; essentially all dermatologicals, like acne preparations, such as benzoyl peroxide, erythromycin benzoyl peroxide, clindamycin phosphate, 5,7-dichloro-8-hydroxyquinoline, and the like; anti-inflammatory agents, such as alclometasone dipropionate, betamethasone valerate, and the like; burn relief ointments, such as o-amino-p-toluene sulfonamide monoacetate, and the like; dermatitis relief agents, such as the active steroid amcinonide, diflorasone diacetate, hydrocortisone, and the like; diaper rash relief agents, such as methylbenzethonium chloride, and the like; emollients and moisturizers, such as mineral oil, PEG-4 dilaurate, lanolin oil, petrolatum, mineral wax, and the like; fungicides, such as butocouazole nitrate, haloprogin, clotrimazole, and the like; herpes treatment drugs, such as O-[(2-hydroxymethyl)-methyl]guanine; pruritic medications, such as alclometasone dipropionate, betamethasone valerate, isopropyl myristate MSD, and the like; psoriasis, seborrhea, and scabicide agents, such as anthralin, methoxsalen, coal tar, and the like; steroids, such as 2-(acetyloxy)-9-fluoro-1′,2′,3′,4′-tetrahydro-11-hydroxypregna-1,4-dieno-[16,17-b]naphthalene-3,20-dione and 21-chloro-9-fluoro-1′,2′,3′,4′-tetrahydro-11b-hydroxypregna-1,4-dieno-[16,17-b]naphthalene-3,20-dione. Any other medication capable of topical administration, like skin protectants, such as allantoin, and antiacne agents, such as salicylic acid, also can be incorporated in a composition of the present invention in an amount sufficient to perform its intended function. Other topically applied compounds are listed in Remington's Pharmaceutical Sciences, 17th Ed., Mack Publishing Co., Easton, Pa. (1985), pages 773-791 and pages 1054-1058 (hereinafter Remington's), incorporated herein by reference.

The present compositions also can contain one or more compounds useful for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide or tocopheryl nicotinate), their isomers, esters, salts and derivatives, and mixtures thereof. The compositions can contain from about 0.0001% to about 15%, about 0.01% to about 10%, about 0.1% to about 5%, or about 0.2% to about 2%, of an oil control agent.

A matte finish salicylic acid lotion is provided below as Example 5.

### EXAMPLE 5

### Matte Finish Anti-Acne Emulsion

| Ingredients (INCI Names) | Wt % |
|---|---|
| Water | 69.50 |
| Methyl Methacrylate/Glycol Dimethacrylate | 8.00 |
| Crosspolymer | |
| Caprylic/Capric Triglyceride | 7.36 |
| Dimethicone | 4.41 |
| Cetearyl Alcohol | 2.82 |
| Glycerin | 2.76 |
| Glyceryl Stearate | 0.92 |
| PEG-100 Stearate | 0.92 |
| Benzyl Alcohol | 0.92 |
| Phenoxyethanol | 0.83 |
| Salicylic Acid | 0.50 |
| Ceteareth-20 | 0.49 |
| Myristyl Myristate | 0.37 |
| Sodium Hydroxide | 0.14 |
| Xanthan Gum | 0.06 |

The topically active compound also can be a plant extract or a natural oil. Nonlimiting plant extracts are those obtained from alfalfa, aloe vera, amla fruit, angelica root, anise seed, apple, apricot, artichoke leaf, asparagus root, banana, barberry, barley sprout, bee pollen, beet leaf, bilberry fruit, birch leaf, bitter melon, black currant leaf, black pepper, black walnut, blueberry, blackberry, burdock, carrot, cayenne, celery seed, cherry, chickwood, cola nut, corn silk, cranberry, dandelion root, elderberry, eucalyptus leaf, flax oil powder, ginger root, gingko leaf, ginseng, goldenrod, goldenseal, grape, grapefruit, guava, hibiscus, juniper, kiwi, kudzu, lemon, licorice root, lime, malt, marigold, myrrh, olive leaf, orange fruit, orange peel, oregano, papaya fruit, papaya leaf, passion fruit, peach, pear, pine bark, plum, pomegranate, prune, raspberry, rhubarb root, rosemary leaf, sage leaf, spearmint leaf, St. John's wart, strawberry, sweet cloves, tangerine, violet herb, watercress, watermelon, willow bark, wintergreen leaf, witch hazel bark, yohimbe, and yucca root. An example of a natural oil is rice bran oil.

The present compositions can contain one or more pigments. The pigments can be any cosmetically acceptable pigment, including organic, inorganic, or mixtures thereof. The composition can contain 0% to about 30%, or about 0.1 to about 30%, by weight, of a pigment. The amount of pigment is related to the desired final color of the composition.

Examples of inorganic pigments include, but are not limited to, iron oxides (yellow, brown, red, black), ultramarines, chromium hydroxide green, chromium oxide, titanium dioxide, ferric ferrocyanide, ferric ammonium ferrocyanide, and mixtures thereof.

Organic pigments include, but arc not limited to, natural colorants, synthetic colorants, and polymeric colorants. Nonlimiting examples arc aromatic compounds such as azo, triphenylmethane, indigo, anthraquinone, and xanthine dyes, referred as D&C and FD&C pigments. Another class of organic pigments is lakes, such as D&C and FD&C lakes and blends and their combinations.

As an example of the current invention, a tinted matte finish cream is illustrated as Example 6.

### EXAMPLE 6

### Tinted Matte Finish Cream

| Ingredients (INCI Names) | Wt % |
|---|---|
| Water | 69.62 |
| Methyl Methacrylate/Glycol Dimethacrylate | 8.00 |
| Crosspolymer | |
| Caprylic/Capric Triglyceride | 7.36 |
| Dimethicone | 3.94 |
| Cetearyl Alcohol | 2.82 |
| Glycerine | 2.76 |
| Glyceryl Stearate | 0.92 |
| PEG-100 Stearate | 0.92 |
| Benzyl Alcohol | 0.92 |
| Phenoxyethanol | 0.83 |
| Ceteareth-20 | 0.49 |
| Magnesium Aluminum Silicate | 0.46 |
| Myristyl Myristate | 0.37 |
| CI 77491 | 0.15 |
| CI 77492 | 0.15 |
| Disodium EDTA | 0.10 |
| Titanium Dioxide | 0.09 |
| CI 77499 | 0.08 |
| Xanthan Gum | 0.02 |

The following additional ingredients typically are included in a present composition. Each of these ingredients, and any other ingredient, is present in a sufficient amount to perform its intended function, without adversely affecting the efficacy of the polymeric microparticles with respect to imparting a matte finish to the skin.

For example, a present composition can contain a surfactant. The surfactant can be an anionic surfactant, a cationic surfactant, a nonionic surfactant, or a compatible mixture of surfactants. The surfactant also can be an ampholytic or amphoteric surfactant, which have anionic or cationic properties depending upon the pH of the composition.

A present composition also can contain a hydrotrope. A hydrotrope is a compound that has an ability to enhance the water solubility of other compounds. Specific examples of hydrotropes include, but are not limited to, sodium cumene sulfonate, ammonium cumene sulfonate, ammonium xylene sulfonate, potassium toluene sulfonate, sodium toluene sulfonate, sodium xylene sulfonate, toluene sulfonic acid, and xylene sulfonic acid. Other useful hydrotropes include sodium polynaphthalene sulfonate, sodium polystyrene sulfonate, sodium methyl naphthalene sulfonate, sodium camphor sulfonate, and disodium succinate.

A present composition also can contain an additional organic solvent. The solvent can be a water-soluble organic compound containing one to six, and typically one to three, hydroxyl groups, e.g., alcohols, diols, triols, and polyols. Specific examples of solvents include, but are not limited to, methanol, ethanol, isopropyl alcohol, n-butanol, n-propyl alcohol, ethylene glycol, propylene glycol, glycerol, diethylene glycol, dipropylene glycol, tripropylene glycol, hexylene glycol, butylene glycol, 1,2,6-hexanetriol, sorbitol, PEG-4, 1,5-pentanediol, similar hydroxyl-containing compounds, and mixtures thereof. The solvent also can be water or an aprotic solvent, e.g., dimethyl sulfoxide or tetrahydrofuran.

A present composition also can contain a thickening or gelling agent. A thickening or gelling agent can be, for example, a polymer that is water soluble or that generates a colloidal solution in water. A thickening or gelling agent, therefore, can be, for example, polymers or copolymers unsaturated carboxylic acids or unsaturated esters, polysaccharide derivatives, gums, colloidal silicates, polyethylene glycols (PEG) and their derivatives, polyvinylpyrrolidones and their derivatives, polyacrylamides and their derivatives, polyacrylonitriles, hydrophilic silica gels, or mixtures thereof.

Specific thickening or gelling agents can be, for example, acrylic and/or methacrylic polymers or copolymers, vinylcarboxylic polymers, polyglyceryl acrylates or methacrylates, polyacrylamides derivatives, cellulose or starch derivatives, chitin derivatives, alginates, hyaluronic acid and its salts, chonodroitin sulphates, xanthan, gellan, Rhamsan, karaya or guar gum, carob flour, and colloidal aluminum magnesium silicates of the montmorillonite type.

Additional thickening or gelling agents include vinylcarboxylic polymers sold under the tradename CARBOPOL® (Lubrizol), acrylic acid/ethyl acrylate copolymers, acrylic acid/stearyl methacrylate copolymers, carboxymethylcellulose, hydroxymethylcellulose, hydroxypropylcellulose, microcrystalline cellulose, hydroxypropyl guar, colloidal hectorites, bentonites, and the like.

Other classes of optional ingredients included in a present composition can be, but not limited to, pH adjusters, chelating agents, preservatives, buffering agents, foam stabilizers, opacifiers, and similar classes of ingredients known to persons skilled in the art. Specific optional ingredients include inorganic phosphates, sulfates, and carbonates as buffering agents; EDTA and phosphates as chelating agents; and acids and bases as pH adjusters. In preferred embodiments, the matte finish composition is free of a coloring agent.

Nonlimiting examples of basic pH adjusters are ammonia; mono-, d and tri-alkyl amines; mono-, di-, and tri-alkanolamines; alkali metal and alkaline earth metal hydroxides; and mixtures thereof. Specific, nonlimiting examples of basic pH adjusters are ammonia; sodium, potassium, and lithium hydroxide; monoethanolamine; triethylamine; isopropanolamine; diethanolamine; and triethanolamine. Examples of acidic pH adjusters are the mineral acids and organic carboxylic acids. Nonlimiting examples of mineral acids are citric acid, hydrochloric acid, nitric acid, phosphoric acid, and sulfuric acid.

The present compositions can include other ingredients traditionally included in cosmetic, dermatological, medicinal, and other such compositions. These ingredients include, but are not limited to, fragrances, preservatives, antioxidants, detackifying agents, and similar types of compounds. The ingredients are included in the composition in an amount sufficient to perform their intended function.

In particular, a present composition can be a lotion; makeup preparation, like makeup foundations; skin care preparation, like hand lotion, vanishing cream, night cream, sunscreen, body lotion, facial cream, clay mask, moisturizing lotion, make-up remover, antiacne preparation, antiaging preparation, and sebum control; analgesic and cortisonal steroid creams and preparations; insect repellants; and facial masks and revitalizers.

A composition of the present invention is topically applied to the skin as needed. Typically, the composition is topically applied to the skin one to four times per day. However, application of a present composition can be more or less frequent as prescribed, required, or desired. The present compositions are applied to the skin by spraying or rubbing. The preferred route of administration is rubbing onto the skin with a soft massage to ensure intimate contact with the skin.

A second skin care composition can be applied over a composition of the present invention, for example, to preserve the integrity of the second composition, increase the wearability of the second composition, or to prevent sebum from coming in contact with the second composition. Non-limiting examples of such compositions include, but are not limited to, sunscreen products, sunless skin tanning products, hair products, fingernail products, moisturizing creams, skin benefit creams and lotions, softeners, day lotions, gels, ointments, foundations, night creams, cheek colors, cleansers, toners, masks, and similar cosmetic and topical therapeutic products.

Obviously, many modifications and variations of the invention as hereinbefore set forth can be made without departing from the spirit and scope thereof and, therefore, only such limitations should be imposed as are indicated by the appended claims.

1. A matte finish composition comprising about 1% to about 10%, by weight, polymeric microparticles having an adsorption capacity of at least 2 grams of oil per gram of polymeric microparticles.

2. The composition of claim 1 wherein the polymeric microparticles are selected from the group consisting of a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, and mixtures thereof.

3. The composition of claim 1 wherein the polymeric microparticles comprise methyl methacrylate/glycol dimethacrylate crosspolymer.

4. The composition of claim 1, wherein the polymeric microparticles are loaded with one or more cosmetic, skin care, or therapeutic agent.

5. The composition of claim 4, wherein the agent comprises cyclomethicone, dimethicone, mineral oil, petrolatum, retinol, a retinal, retinyl acetate, retinyl palmitate, retinoic acid, retinyl propionate, retinyl linoleate, dehydroretinol, eretinate, eretrin, motretinide, glycolic acid, kojic dipalmitate, hydroquinone, or a mixture thereof.

6. The composition of claim 5 wherein the one or more agent is loaded onto the polymeric microparticles in an amount to provide loaded polymeric microparticles comprising about 0.01% to about 80%, by weight, of the one or more agent.

7. The composition of claim 4 wherein the one or more agent comprises an antiacne agent selected from the group consisting of salicylic acid, sulfur, resorcinol, tretinoin, adapalene, dapsone, clindamyacin, benzamyacin, azelaic acid, benzoyl peroxide, and mixtures thereof.

8. The composition of claim 7 wherein the amount of the antiacne agent is loaded onto the polymeric microparticles in an amount to provide loaded microparticles comprising about 0.01% to about 50%, by weight, of the antiacne agent.

9. The composition of claim 1 comprising about 1% to about 8%, by weight, polymeric microparticles.

10. The composition of claim 1 comprising about 4% to about 8%, by weight, polymeric microparticles.

11. The composition of claim 1 wherein the composition is an oil-in-water emulsion.

12. The composition of claim 1 wherein the composition is a water-in-oil emulsion.

13. The composition of claim 1 wherein the composition is an aqueous gel.

14. The composition of claim 1 wherein the composition is an anhydrous gel.

15. The composition of claim 1 further comprising a topically applied compound selected from the group consisting of a pesticide, a drug, a therapeutic agent, a skin lightening agent, a deodorant, a skin conditioner, an antioxidant, an insect repellant, a counterirritant, a vitamin, a plant extract, a natural oil, a steroid, an antibacterial compound, an antifungal compound, an anti-inflammatory compound, a topical anesthetic, an epidermal lipid replacement, a sunscreen, an optical brightener, a dermatitis or skin disease medication, and mixtures thereof.

16. The composition of claim 15 wherein the topically applied compound is loaded onto polymeric microparticles, is present in the composition in free form, or both.

17. The composition of claim 1 wherein the composition comprises one or more pigment.

18. The composition of claim 17 wherein the composition comprises about 0.1% to about 30%, by weight, of the one or more pigment.

19. A method of imparting a matte finish to skin comprising topically applying a composition of claim 1 to the skin.

20. The method of claim 19 wherein the skin has a matte finish appearance for at least 6 hours.

21. The method of claim 19 wherein a second skin care composition is topically applied over the composition of claim 1.

22. A method of adsorbing sebum from skin comprising topically applying a composition of claim 1 to the skin.
